(19) United States
(12) Patent Application Publication
(10) Pub. No.: US 2017/0016886 A1
(43) Pub. Date: Jan. 19, 2017

(54) INDUCED PLURIPOTENT STEM CELL MODEL OF NOONAN SYNDROME AND USE THEREOF

(71) Applicant: KOREA ADVANCED INSTITUTE OF SCIENCE AND TECHNOLOGY, Daejeon (KR)

(72) Inventors: Yong-Mahn HAN, Daejeon (KR); Young Hee Ju, Daejeon (KR)

(73) Assignee: KOREA ADVANCED INSTITUTE OF SCIENCE AND TECHNOLOGY, Daejeon (KR)

(21) Appl. No.: 15/221,217

(22) Filed: Jul. 27, 2016

Related U.S. Application Data
(63) Continuation of application No. PCT/KR2014/000880, filed on Jan. 29, 2014.

(30) Foreign Application Priority Data
Jan. 28, 2014 (KR) 10-2014-0010398

(51) Int. Cl. G01N 33/50

(52) U.S. Cl. CPC G01N 33/5073; G01N 33/5058; G01N 33/5023; G01N 33/5026; G01N 2800/385

(57) ABSTRACT

The present invention relates to an induced pluripotent stem cell (iPSC) model of Noonan syndrome, a preparation method thereof, and uses to study of the pathogenesis of Noonan syndrome and a therapeutic agent screening method. Particularly, induced pluripotent stem cells from dermal fibroblasts of a Noonan syndrome-patient (NS-iPSCs) were generated, and differentiated into embryoid bodies (EBs), neural rosettes and neural cells. These iPSCs exhibited the normal morphology while showed reduced differentiation potency compare to control cell lines. NS-iPSCs were developed into embryoid bodies and neural rosettes by naturally and chemically directed differentiation. Interestingly, embryoid bodies and neural rosettes induced via chemically directed differentiation exhibited normal morphology and expressed ectoderm, neural rosettes and neural marker genes similar to normal cells. Thus, the cellular model can be useful in analytical research to understand pathogenesis of Noonan syndrome and establish screening method of the therapeutic agent.

### CROSS-REFERENCE TO RELATED APPLICATIONS

This application is a continuation of PCT/KR2014/000880, filed Jan. 29, 2014, which claims the benefit of Korean Patent Application No. 10-2014-0010398, filed Jan. 28, 2014, the contents of each of which are incorporated herein by reference.

### BACKGROUND OF THE INVENTION

1. Field of the Invention

The present invention relates to an induced pluripotent stem cell (iPSC) model of Noonan syndrome, a preparation method thereof, and uses to study of the pathogenesis of Noonan syndrome and a therapeutic agent screening method.

2. Description of the Related Art

Noonan syndrome is a genetic disease caused by the excessive activity of Ras-MAPK signal transduction system, which was reported first by J. A. Noonan in 1983. Noonan syndrome was first confused with Turner syndrome due to the similar external symptoms between the two, however it displays a normal karyotype unlike Turner syndrome and is equally developed in both men and women, which separates Noonan syndrome from Turner syndrome.

Symptoms and physical manifestations in their degrees and ranges vary from Noonan syndrome patients. Typical symptoms are characteristic looking and disorders in growth and development. As mentioned above, degrees and ranges of symptoms vary, so that patients are all different from those who can live a normal life to those who display mental disorder or developmental disorder. Major symptoms of Noonan syndrome reported are listed in Table 1 below, which are mainly shown in ectoderm and mesoderm related body parts rather than in endoderm related body parts.

TABLE 1
Major symptoms of Noonan syndrome

| | | |
|---|---|---|
| Appearance | Face | long and narrow face with pointy top, |
| features | | triangle shape, noticeable philtrum, |
| | | and wrinkled skin |
| | Mouth | deep philtrum, wide upper lip (95%), |
| | | arch-shaped palate (45%), and small |
| | | chin (25%) |
| | Neck | webbed neck, cystic lymphangioma, |
| | | short neck |
| Growth and | Adolescence | short stature (50~70%) |
| developmental | Infancy | developmental disability (40%), |
| disability | | developmental delay (26%), learning |
| | | disorder (15%), |
| | | delayed language development (20%), |
| | | and slight mental retardation (35%) |

| | |
|---|---|
| Blood disorder and | hands and feet lymphedema (neonatal |
| lymphatic | period), blood coagulation delay, |
| dysfunction | leukemia |
| Urogenital disorder | cryptorchidism (60~80%), kidney |
| | deformity (10%) |
| Ocular | ptosis (95%), strabismus (50~60%), |
| manifestations | ocular hypertelorism, myopia and |
| | hyperopia (60~70%), and nystagmus |
| | (10%) |
| Musculoskeletal | thoracic deformity (90%), scoliosis |
| disorder and | (10~15%), foot deformity (10~15%), |
| teeth developmental | cubitus valgus (50%), cervical fusion |
| disability | (2%), and teeth malocclusion |
| Neurological | convulsion (10%), hypotonia, flexible |
| disorder | joint, rarely brain deformity |
| Cardiac anomaly | abnormal electrocardiogram (90%), |
| | congenital heart malformation (68%), |
| | pulmonary valve related deformity |
| | (20~50%), and hypertrophic |
| | cardiomyopathy (20~30%) |

A PTPN11 gene that causes Noonan syndrome is located in 12^{th} chromosome. PTPN11 encodes SHP-2 (Src-homology domain 2 containing tyrosine phosphatase). The SHP2 protein is involved in activation of Ras-MAPK pathway via phosphorylation of ERK. More precisely, once the protein is activated by the conjugation between the ligand and the receptor such as FGFR, EGFR, HGFR, and MET, it independently activates Ras, or dependently transmits signals through GRB2 and SOS1, resulting in the phosphorylation of ERK that affects Ras-MAPK pathway (Yoko Aoki et al., (2008) Human mutation, 29(8), 992-1006).

Ras-MAPK signal transduction system is involved in various biological functions including cell proliferation, migration, survival, cell fate determination, cytoskeletal rearrangement, metabolism, and senescence, etc. It is also involved in morphological determination, organogenesis, and neural growth.

The activation of Ras-MAPK signal transduction system is induced by the conjugation of a ligand such as cytokine, growth factor, or hormone to a receptor. When a SHP2 protein as a mediator of Ras-MAPK signaling pathway is mutated by single nucleotide substitution of PTPN11, the activation of the pathway is abnormally induced even without the conjugation of the ligand to the receptor. As a result, normal development and differentiation of cell would be affected to cause functional disorders.

To construct a method for screening of therapeutic agents and studying about pathogenesis of Noonan syndrome, transgenic mouse models have been established to reenact noonan syndrome. However, developmental mechanisms of Noonan syndrome have not been disclosed yet. In addition, studies on a therapeutic agent or treatment method for Noonan syndrome are still insufficient. Therefore, it is requested to disclose the patient-specific developmental mechanism and to develop a therapeutic agent for the disease.

Embryonic stem cells (ESCs) are the cell of undifferentiated stage just before the differentiation into each cell that forms an organ, which can be obtained from embryo, fetus, and adult tissues. The ESCs are capable of self renewing in undifferentiated state and have pluripotency so that it can be differentiated into various kinds of cells by a certain stimulus. So, stem cells can differentiate into a specific cell by a certain stimulus (environment). Unlike fully differentiated cells, stem cells are capable of self-renewal via cell division to maintain undifferentiated state, suggesting that stem cell has the ability of proliferation and expansion. The stem cell can be differentiated into another cell when different differentiation stimulus (environment) is given, suggesting that stem cell has plasticity.

Human pluripotent stem cells (hPSCs) including induced pluripotent stem cells (iPSCs) can give rise to specialized types of cells in body. When iPSCs were differentiated in vitro, not only therapeutic potential due to the low risk of immune rejection but also usability in understanding the developmental mechanism of a complex disease in the early organogenesis were confirmed (Muotri, A. R. (2009) Epilepsy Behav 14 Suppl 1: 81-85; Marchetto, M. C., B. Winner, et al. (2010) Hum Mol Genet 19(R1): R71-76).

Up to date, reports have been made to confirm that when iPSCs derived from patients with various genetic diseases were differentiated into the disease-associated cell kinds directly, they demonstrated the disease-specific phenotypes (Park, I. H. et al. Cell 134, 877-886 (2008); Tiscornia, G. et al. Nature medicine 17, 1570-1576 (2011)). Such patient-derived iPSCs can be differentiated into the tissue- or organ-specific cells related to disease and thereby they are expected to be used for the disclosure of the concrete mechanism of the disease and for the screening of a therapeutic agent thereof.

Thus, the present inventors tried to establish a stem cell model usable for the study of Noonan syndrome.

As a result, the inventors succeeded in establishing induced pluripotent stem cells (iPSCs) from fibroblasts of a Noonan syndrome patient. Generated iPSCs were differentiated into embryoid bodies (EBs) and neural rosettes. iPSCs derived from noonan syndrome (NS-iPSCs) displayed similar morphology compare to normal iPS cells, while NS-EBs had reduced differentiation potency. The inventors also induced the natural differentiation and chemically directed differentiation of NS-iPSCs into embryoid bodies and neural rosettes. As a result, the embryoid bodies and neural rosettes induced via chemically directed differentiation displayed normal morphology similar to that of normal cells and significantly expressed marker genes of ectoderm, neural rosettes, and neural cells. Therefore, the cellular modeling of Noonan syndrome using the induced pluripotent stem cells (iPSCs) of the present invention can be effectively used for the study of Noonan syndrome and for the screening of a therapeutic agent thereof, leading to the completion of the invention.

### SUMMARY OF THE INVENTION

It is an object of the present invention to provide novel induced pluripotent stem cells (iPSCs) displaying the same characteristics as Noonan syndrome patient cells and a method for using the same in the study for the pathogenesis of Noonan syndrome and for the screening of a therapeutic agent for the disease.

To achieve the above object, the present invention provides a method for preparing an induced pluripotent stem cell (iPSC) model of Noonan syndrome in vitro comprising the following steps:

i) inducing induced pluripotent stem cells (iPSCs) from the fibroblasts separated from a Noonan syndrome patient in vitro; and

ii) collecting the induced pluripotent stem cells (iPSCs) induced in step i).

The present invention also provides a Noonan syndrome iPSC model prepared by the method above.

The present invention further provides a method for using the iPSCs above as the Noonan syndrome model, which comprises the following steps:

i) inducing the differentiation of the iPSCs into embryoid bodies (EBs) or neural cells; and

ii) analyzing the characteristics of the embryoid bodies or neural cells differentiated in step i).

The present invention also provides a method for screening a therapeutic agent candidate for Noonan syndrome comprising the following steps:

i) preparing an iPSC model, and obtaining embryoid bodies or neural cells differentiated from the prepared iPSC model;

ii) treating the test compound or the test composition to the iPSC model, the embryoid bodies, or the neural cells of step i);

iii) analyzing the characteristics of the iPSC model, the embryoid bodies, or the neural cells treated in step ii); and

iv) comparing the results of the analysis of step iii) with the non-treated control.

The present invention also provides a use of the Noonan syndrome iPSC model prepared by the method above.

In addition, the present invention provides a use of the iPSCs above as a Noonan syndrome model, which comprises the following steps:

i) inducing the differentiation of the iPSCs into embryoid bodies or neural cells; and

ii) analyzing the characteristics of the embryoid bodies or neural cells differentiated in step i).

### Advantageous Effect

The stem cell model using the induced pluripotent stem cells (iPSCs) derived from the fibroblasts of Noonan syndrome patient can be differentiated into embryoid bodies and neural cells. At this time, the differentiated neural cells can significantly express ectoderm, neural rosette, and neural marker genes but are reduced expression of NR2F1 gene, so that the cellular model of the invention can be effectively used for the study of the pathogenesis of Noonan syndrome and for the study of establishing a screening method of a therapeutic agent for Noonan syndrome.

### BRIEF DESCRIPTION OF THE DRAWINGS

The application of the preferred embodiments of the present invention is best understood with reference to the accompanying drawings, wherein:

FIG. 1 presents the morphology of the induced pluripotent stem cells (iPSCs) generated from the dermal fibroblasts of a noonan syndrome-patient.

FIG. 2 presents the confirmation of the mutation of a causing gene of Noonan syndrome in the iPSCs derived from a noonan syndrome-patient (NS-iPSCs).

FIG. 3 presents the result of bisulfite sequencing in undifferentiated NS-iPSCs to investigate the demethylation in promoter region of pluripotent genes.

FIG. 4 presents the normal karyotype in NS-iPSCs.

FIG. 5 presents the result of alkaline phosphatase staining (AP staining) to confirm whether or not the pluripotency of NS-iPSCs are maintained.

FIG. 6 presents expressions of the pluripotent marker genes to confirm the pluripotency of NS-iPSCs.

FIG. 7 presents the expressions of the stem cell markers including OCT4, NANOG, SOX2, SSEA4, Tra-1-81, and Tra-1-60 in the NS-iPSC #4.

FIG. 8 presents the expressions of the stem cell markers including OCT4, NANOG, SOX2, SSEA4, Tra-1-81, and Tra-1-60 in the NS-iPSC #5.

FIG. 9 presents the teratoma formation investigated for the confirmation of in vivo differentiation potency in the NS-iPSC #4.

FIG. 10 presents the teratoma formation investigated for the confirmation of in vivo differentiation potency in the NS-iPSC #5.

FIG. 11 is a schematic diagram illustrating the natural differentiation of EBs and neural rosettes (neuroectoderm) from NS-iPSCs.

FIG. 12 illustrates the cell morphology of EBs and neural rosettes naturally differentiated from NS-iPSCs.

FIG. 13 illustrates the decrease of the stem cell related gene expression level in EBs naturally differentiated from NS-iPSCs.

FIG. 14 illustrates the changes in the signal transduction related gene expression level in EBs naturally differentiated from NS-iPSCs.

FIG. 15 illustrates the increase of the signal transduction related protein phosphorylation level in EBs naturally differentiated from NS-iPSCs.

FIG. 16 illustrates the decrease of the neural marker gene expression level in neural rosettes naturally differentiated from NS-iPSCs.

FIG. 17 illustrates the quantitative decrease of the neural marker gene expression level in neural rosettes naturally differentiated from NS-iPSCs.

FIG. 18 is a schematic diagram illustrating the chemically directed differentiation of NS-iPSCs into EBs and neural rosettes.

FIG. 19 presents the morphology of EBs and neural rosettes chemically differentiated NS-iPSCs.

FIG. 20 presents the morphology of EBs, neural rosettes, neural precursors, and neural cells chemically differentiated from NS-iPSCs.

FIG. 21 illustrates the expressions of ectoderm, neural rosette, and neural cell marker genes in neural rosettes chemically differentiated from NS-iPSCs.

FIG. 22 presents the quantitative decrease of the NR2F1 gene expression in neural rosettes chemically differentiated from NS-iPSCs.

### DESCRIPTION OF THE PREFERRED EMBODIMENTS

Hereinafter, the present invention is described in detail.

The present invention provides a method for preparing an induced pluripotent stem cell (iPSC) model of Noonan syndrome in vitro comprising the following steps:

i) inducing induced pluripotent stem cells (iPSCs) from the fibroblasts separated from a Noonan syndrome patient in vitro; and

ii) collecting the induced pluripotent stem cells (iPSCs) induced in step i).

In step i), the inducement is accomplished by using the ectopic expressions of pluripotent markers including OCT4, SOX2, KLF4, and c-MYC, but not always limited thereto.

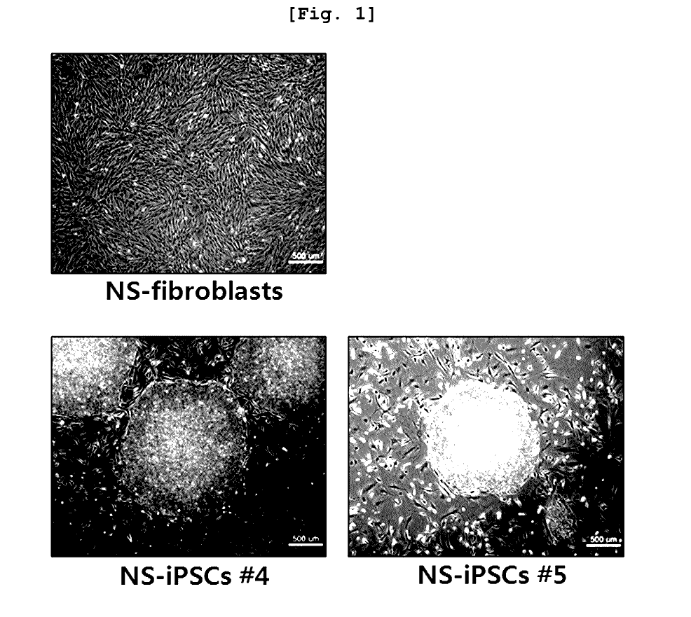

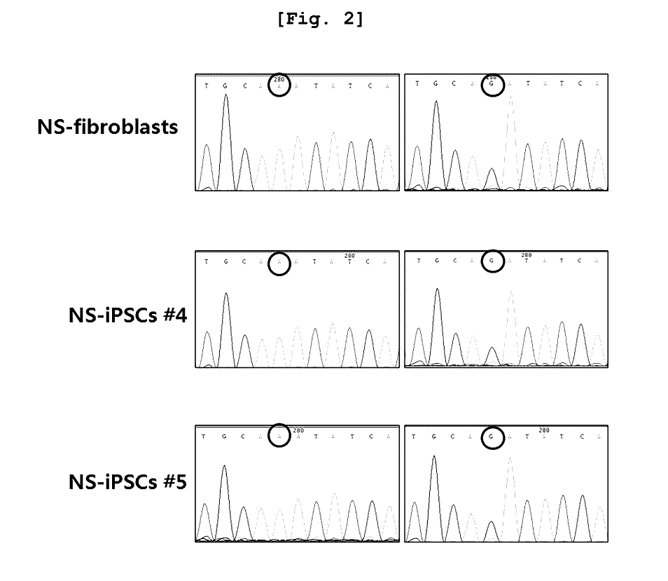

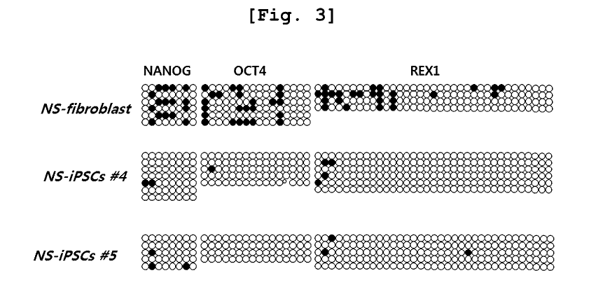

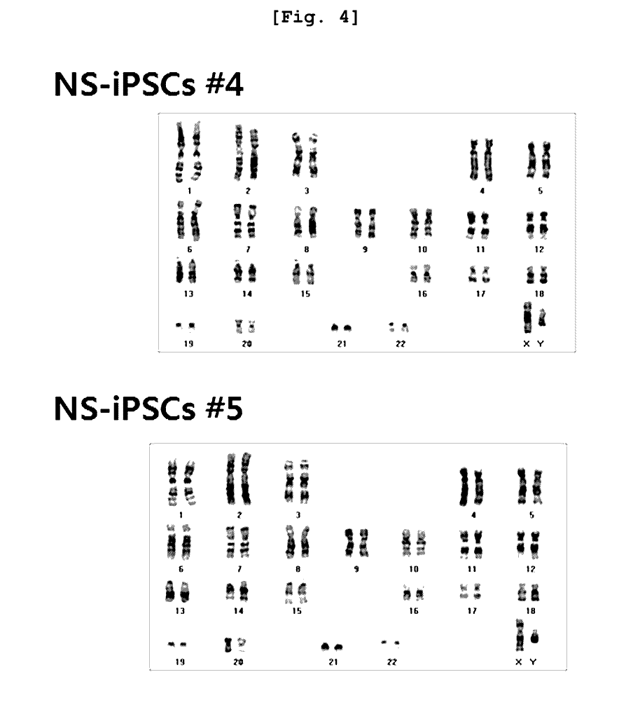

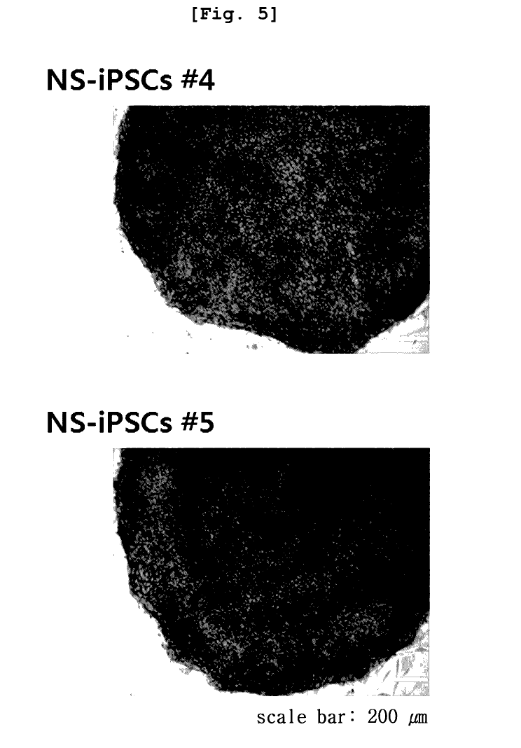

In a preferred embodiment of the present invention, the inventors confirmed the primary clinical symptoms of a Noonan syndrome-patient and the mutation of PTPN11 gene in the fibroblasts originated from the Noonan syndrome patient (see Tables 2 and 3), and also induced the generation of iPSCs (NS-iPSCs) from the fibroblasts above (see FIG. 1). The inventors also confirmed that the NS-iPSCs above could have the causing gene of Noonan syndrome (see FIG. 2), maintained the normal karyotype (see FIG. 4) and remained as undifferentiated status (see FIGS. 3 and 5).

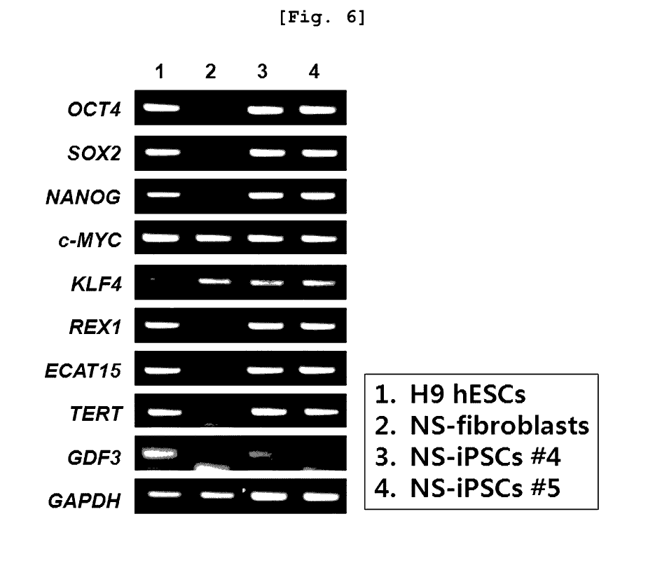

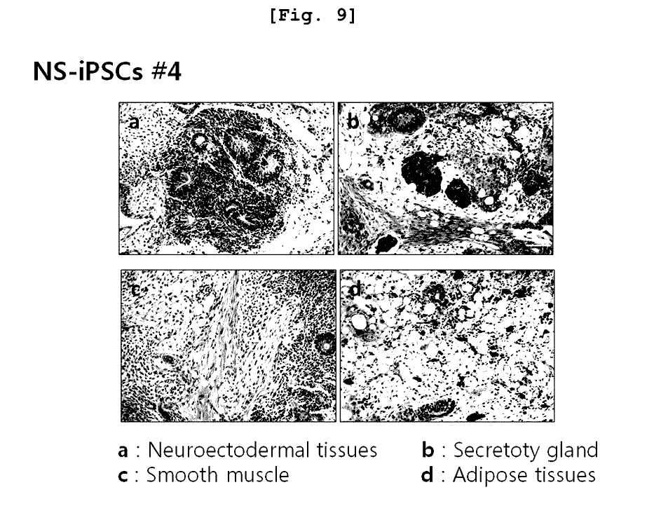

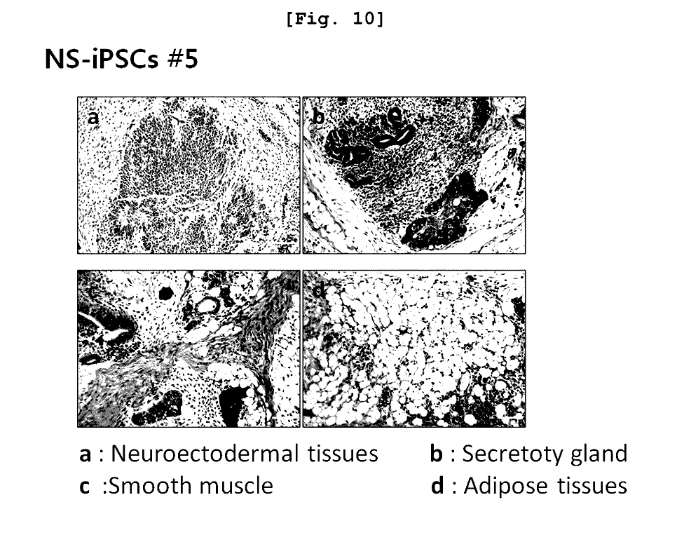

The present inventors investigated the differentiation potency of the NS-iPSCs of the invention. As a result, it was confirmed that the NS-iPSCs expressed the pluripotent marker gene and protein significantly (see FIGS. 6˜8) and formed teratoma in vivo (see FIGS. 9 and 10), suggesting that the NS-iPSCs had pluripotency.

Therefore, the Noonan syndrome-derived iPSC model of the present invention has pluripotency with displaying the same mutation as the Noonan syndrome patient shows, so that the iPSC model of the invention can be effectively used for the study of Noonan syndrome.

The present invention also provides a Noonan syndrome iPSC model prepared by the method above.

The iPSC herein is characterized by one or more characteristics selected from the followings, but not always limited thereto:

i) normal iPSC morphology;

ii) expressing one or more pluripotent marker genes selected from the group consisting of OCT4, SOX2, NANOG, c-MYC, REX1, ECAT15, GDF3, and TERT; and

iii) expressing one or more stemness marker proteins selected from the group consisting of OCT4, SOX2, NANOG, SSEA4, Tra-1-81, and Tra-1-60.

The Noonan syndrome-derived iPSC model of the present invention has pluripotency with displaying the same mutation in the gene derived from fibroblasts of the Noonan syndrome patient, so that the iPSC model of the invention can be effectively used for the study of Noonan syndrome.

The present invention further provides a method for using the iPSCs above as the Noonan syndrome model, which comprises the following steps:

i) inducing the differentiation of the iPSCs into embryoid bodies (EBs) or neural cells; and

ii) analyzing the characteristics of the embryoid bodies or neural cells differentiated in step i).

The inducement herein is achieved by either natural differentiation without any external cues or directed differentiation via treatment of chemicals, but not always limited thereto.

These chemicals used herein can be accepted for the differentiation of stem cells into neural cells, which is exemplified by dorsomorphin and SB431542. More precisely, one of or both of dorsomorphin and SB431542 can be used, but not always limited thereto. Dorsomorphin and SB431542 can be simultaneously or sequentially added to a medium in the course of differentiation.

The EBs herein is characterized by one or more characteristics selected from the followings, but not always limited thereto:

i) normal morphology of cells;

ii) expressing one or more pluripotent marker genes selected from the group consisting of OCT4, SOX2, NANOG, and c-MYC;

iii) increasing BMP signaling genes including one of or both of Id1 and Id2;

iv) increasing the phosphorylation level of one or more BMP signaling proteins selected from the group consisting of p-SMAD1, p-SMAD5, and p-SMAD8;

v) increasing TGF-β signaling genes including one of or both of SMAD2 and SMAD3; and

vi) increasing the phosphorylation level of TGF-β signaling proteins including one of or both of p-SMAD2 and p-SMAD3.

The neural cells herein are characterized by one or more characteristics selected from the followings, but not always limited thereto:

i) normal morphology of cells; and

ii) expressing one or more neural genes selected from the group consisting of PAX6, ZIC1, NESTIN, VIMENTIN, PLZF, HES5, DACH1, TUJ1, ASCL1, and NF1.

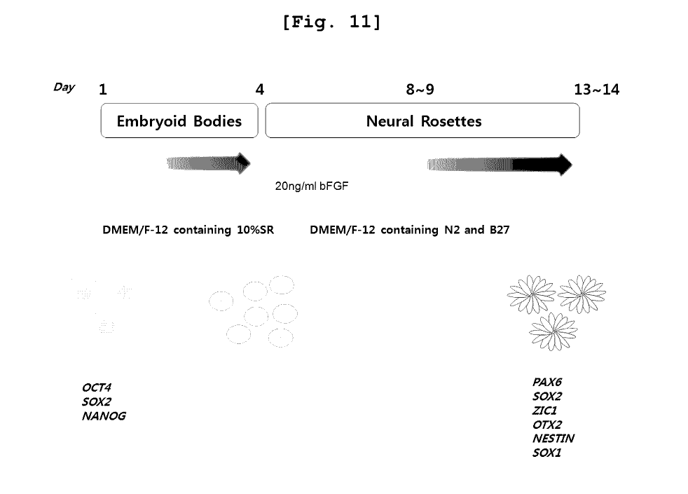

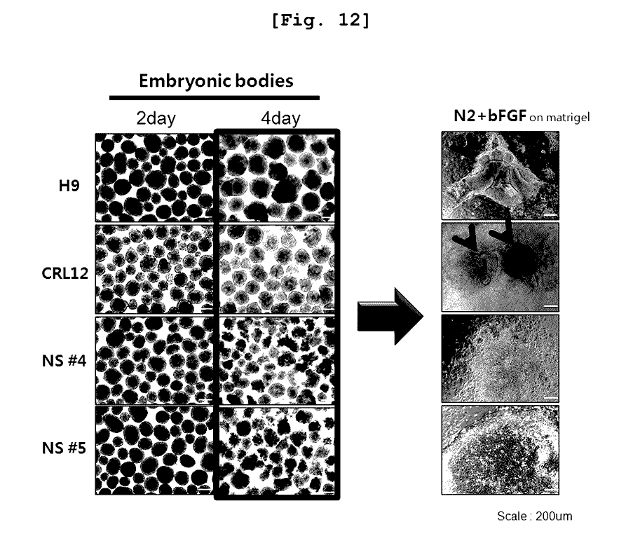

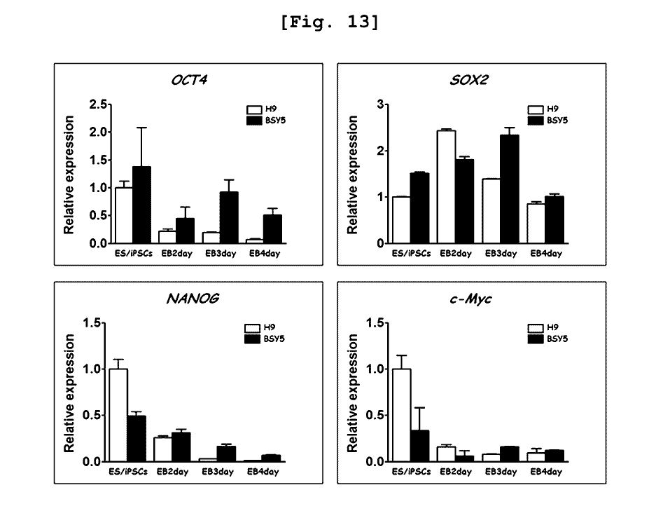

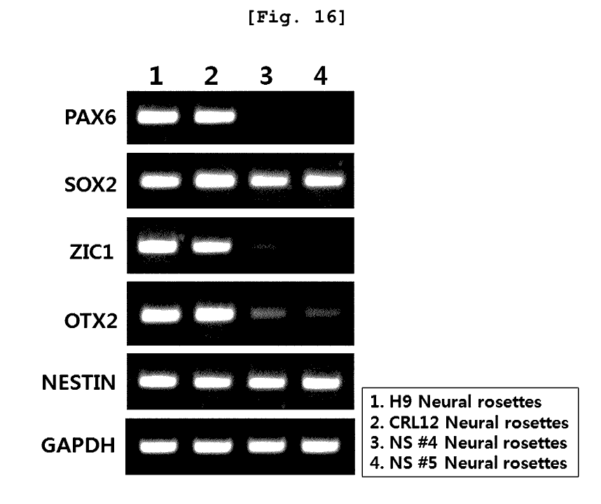

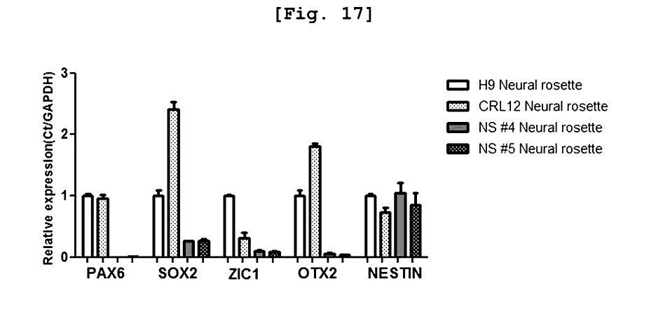

In another preferred embodiment of the present invention, iPSCs derived from the Noonan syndrome patient were naturally differentiated to obtain EBs and neural rosettes (see FIG. 11). The cells displayed normal appearances at 2 days after the differentiation, but after that morphologies of EBs and neural rosette were broken (see FIG. 12). At the same time, the EBs exhibited the decrease of the pluripotent marker genes, the increase of expressions both BMP and TGF-β signaling-related genes and the increase of phosphorylation levels of the BMP signaling-related proteins (see FIGS. 13˜15). The expressions of neural marker genes were not observed in neural rosettes, though (see FIGS. 16 and 17).

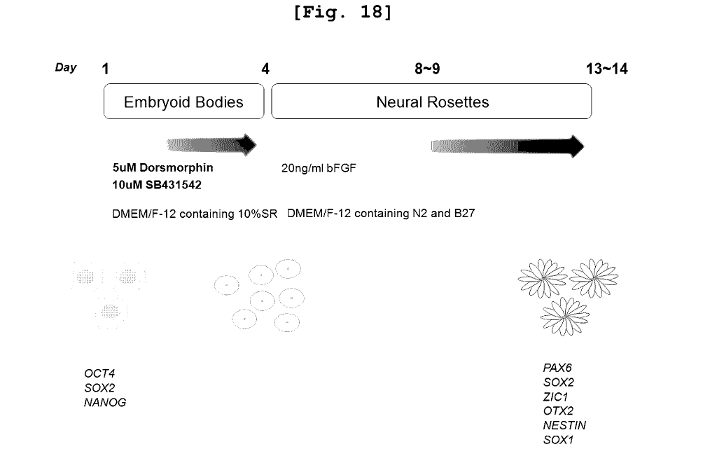

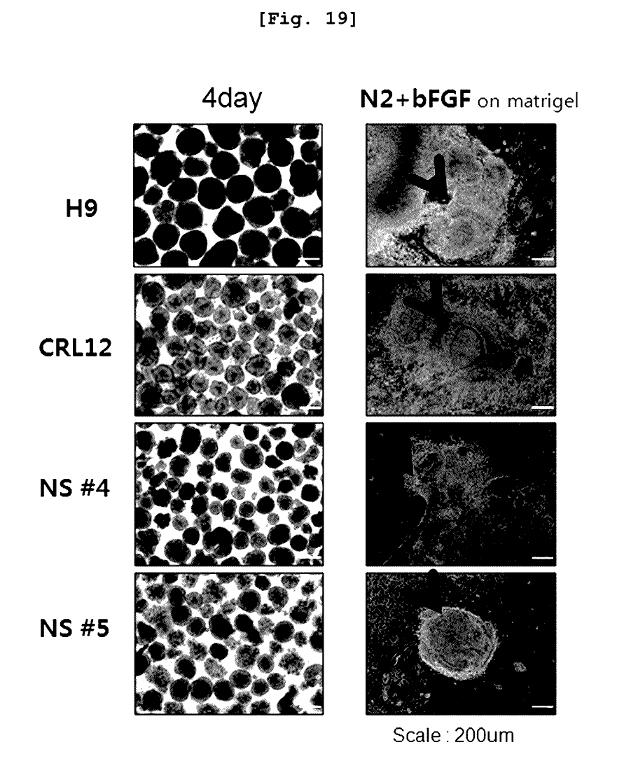

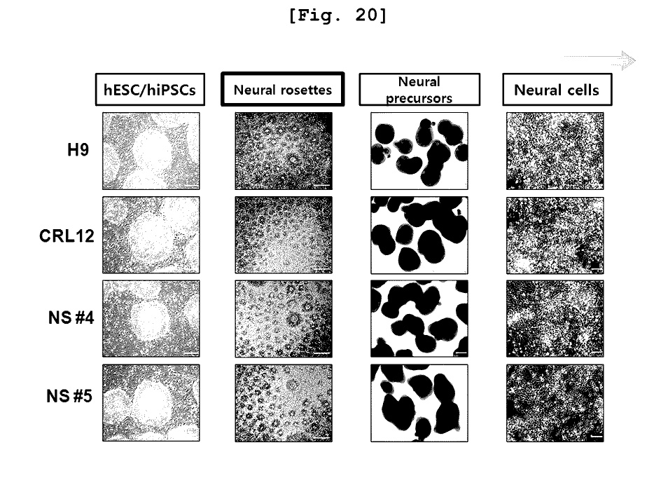

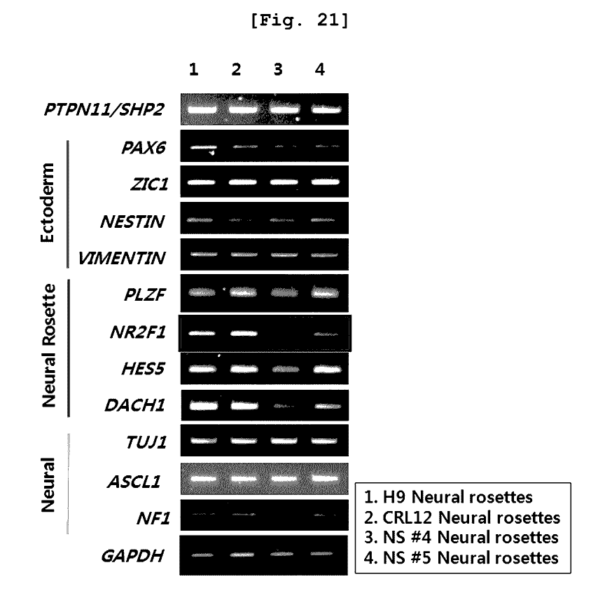

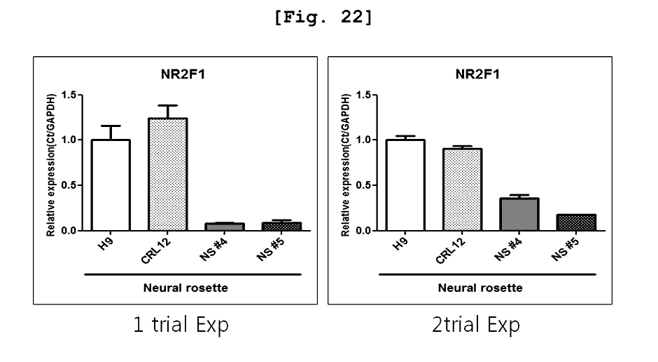

NS-iPSCs were chemically induced into EBs and neural rosettes by stepwise treatment of dorsomorphin and SB431542 (see FIG. 18). The cell morphology of the chemically differentiated EBs and neural rosettes was recovered to a normal shape (see FIGS. 19 and 20) and the expressions of ectoderm, neural rosette, and neural marker genes were adjusted to the nearly normal level. However, the expression of NR2F1, the orphan nuclear receptor which is one of neural rosette genes and whose ligand has not been identified yet, was decreased (see FIGS. 21 and 22). The involvement of this gene in any disease has not been studied yet and the role of this gene in the early developmental stage has not been disclosed, either, suggesting that more studies are needed for this gene.

Therefore, the Noonan syndrome-derived iPSC model of the present invention can be differentiated into EBs and neural cells, so that the cellular model can be effectively used for the study of Noonan syndrome and for the screening of a therapeutic agent thereof

The present invention also provides a method for screening candidate of therapeutic agent for Noonan syndrome comprising the following steps:

i) preparing an iPSC model, and obtaining embryoid bodies or neural cells differentiated from the prepared iPSC model;

ii) treating the test compound or the test composition to the iPSC model, the embryoid bodies, or the neural cells of step i);

iii) analyzing the characteristics of the iPSC model, the embryoid bodies, or the neural cells treated in step ii); and

iv) comparing the results of the analysis of step iii) with the non-treated control.

The inducement herein is achieved by either natural differentiation or directed differentiation with a chemical, but not always limited thereto.

The chemicals used herein can be accepted for the differentiation of stem cells into neural cells, which is exemplified by dorsomorphin and SB431542. More precisely, one of or both of dorsomorphin and SB431542 can be used, but not always limited thereto. Dorsomorphin and SB431542 can be simultaneously or sequentially added to a medium in the course of differentiation.

The EBs herein is characterized by one or more characteristics selected from the followings, but not always limited thereto:

i) normal morphology of cells;

ii) expressing one or more pluripotent marker genes selected from the group consisting of OCT4, SOX2, NANOG, and c-MYC;

iii) increasing BMP signaling genes including one of or both of Id1 and Id2;

iv) increasing the phosphorylation level of one or more BMP signaling proteins selected from the group consisting of p-SMAD1, p-SMAD5, and p-SMAD8;

v) increasing TGF-β signaling genes including one of or both of SMAD2 and SMAD3; and

vi) increasing the phosphorylation level of TGF-β signaling proteins including one of or both of p-SMAD2 and p-SMAD3.

The neural cells herein is characterized by one or more characteristics selected from the followings, but not always limited thereto:

i) normal morphology of cells; and

ii) expressing one or more neural genes selected from the group consisting of PAX6, ZIC1, NESTIN, VIMENTIN, PLZF, HES5, DACH1, TUJ1, ASCL1, and NF1.

The therapeutic agent candidate for Noonan syndrome can be preferably selected among those materials capable of leading the iPSC model and EBs or neural cells similar to level of the normal control. Particularly, a preferable candidate is the one that increases the expression of NP2F1 in neural cells differentiated from the iPSC model of the invention to the similar level of the normal cells, but not always limited thereto.

The EBs and neural cells naturally differentiated from the Noonan syndrome-derived iPSC model of the invention can realize the abnormal morphology and gene expressions, while the chemically induced EBs and neural cells display normal morphology and expressions of neural markers, so that the iPSC model of the invention can be effectively used to screen candidates as therapeutic agent for Noonan syndrome.

The present invention also provides a use of the Noonan syndrome iPSC model prepared by the method above.

In addition, the present invention provides a use of the iPSCs above as a Noonan syndrome model, which comprises the following steps:

i) inducing the differentiation of the iPSCs into embryoid bodies or neural cells; and

ii) analyzing the characteristics of the embryoid bodies or neural cells differentiated in step i).

The naturally differentiated EBs and neural cells induced from the Noonan syndrome-derived iPSC model of the invention can realize the abnormal cell morphology and gene expression characteristically shown in Noonan syndrome, while the chemically induced EBs and neural cells display normal morphology and expressions of neural marker genes, so that the iPSC model of the invention can be effectively used for screening of a therapeutic agent candidate for Noonan syndrome.

Practical and presently preferred embodiments of the present invention are illustrative as shown in the following Examples.

However, it will be appreciated that those skilled in the art, on consideration of this disclosure, may make modifications and improvements within the spirit and scope of the present invention.

### Example 1

### Confirmation of Clinical Symptoms and Mutation of the Causing Gene of Noonan Syndrome

### <1-1> Confirmation of Clinical Symptoms of Noonan Syndrome

To confirm the clinical symptoms of a Noonan syndrome patient, a Noonan syndrome patient was selected and observed clinical symptoms thereof.

Particularly, a Noonan syndrome patient (B.S.Y, male) was notified by Asan Medical Center (Korea), and the representative Noonan syndrome symptoms were observed as shown in Table 2 (Table 2).

TABLE 2
Clinical symptoms of Noonan syndrome

| Feature | Description | Feature | Description |
|---|---|---|---|
| Face | Typical | Congenital heart | ∘ |
| | shape | disease | |
| Neck | Short neck | Hypertrophic | x |
| problem | | cardiomyopathy | |
| | | (HCMP) | |
| Short | ∘ | Pulmonary stenosis | ∘ |
| stature | | | |
| <3 p | ∘ | Intellectual | Borderline |
| | | disability | Intelligence |
| | | | decline |

### <1-2> Confirmation of Mutation of the Causing Gene of Noonan Syndrome

To confirm the mutation of the causing gene of Noonan syndrome, the sequence of PTPN11 gene known as a Noonan syndrome-related gene was investigated in the fibroblasts of a Noonan syndrome patient.

Particularly, after permitted from the review by Institutional Review Board, skin tissue biopsy was performed by punch biopsy method with the Noonan syndrome patient selected in Example <1-1> under the agreement of the patient and his legal representative, and more precisely dermal tissue was obtained from the Noonan syndrome patient. Then, fibroblasts were separated from the obtained dermal tissue, followed by culture in DMEM (Dulbecco's modified Eagle's medium, Welgene, Korea) supplemented with 10% FBS (fetal bovine serum, GIBCO, USA), 1% penicillin (GIBCO, USA), and 1% MEM-NEAA solution (MEM Non-Essential Amino acids Solution; GIBCO, USA). Genomic DNA (gDNA) was extracted from the fibroblasts and the sequence of PTPN11 gene was identified.

As a result, as shown in Table 3, the Noonan syndrome patient showed A922G (N308D) mutation in PTPN11 gene (Table 3).

TABLE 3
Mutation of causing gene of Noonan syndrome

| | | DNA base | Protein amino | Mutation | Reference |
|---|---|---|---|---|---|
| Gene | Location | change | acid change | region | sequence |
| PTPN11 | 12q24.13 | c.922A > G | p.Asn308Asp | Exon 8 | NM_002834.3 |
| | | (AAT→GAT) | (N308D) | PTP | |

### Example 2

### Preparation of Noonan Syndrome-Derived Induced Pluripotent Stem Cells (iPSCs)

<2-1> Inducement of the Development of Noonan Syndrome Patient-Derived iPSCs

The development of Noonan syndrome-derived iPSC (NS-iPSC) was induced by ectopic expression (Takahashi, K et al, Cell 131(5): 861-872, 2007) via the reprogramming factors including OCT4, SOX2, C-MYC, and KLF4.

Particularly, the Noonan syndrome patient-derived fibroblasts obtained in Example <1-2> were cultured in DMEM supplemented with 10% FBS (fetal bovine serum; GIBCO, USA). Then, the fibroblasts were infected with the retrovirus expressing OCT4, SOX2, c-MYC, and KLF4. The infected fibroblasts were loaded on the mouse embryonic fibroblasts (MEF) treated with mitomycin C (AG scientific, USA), and co-cultured in DMEM/F12 (GAIBCO, USA) supplemented with 20% serum replacement (GAIBCO, USA) and 10 ng/ml of bFGF (R&D systems, USA) for 10˜15 days. Then, sub-culture was performed to induce generation of NS-iPSC. The induced NS-iPSCs were observed under phase-contrast microscopy (Olympus, Japan). For comparison with normal control iPSCs (CRL-12) were established from human skin fibroblasts (CRL-2097; American Type Culture Collection; ATCC, USA) by the same manner as described above (Kim et. al, BBRC 424 (2012) 331-337; This is the same as the iPSCs #2 shown in the reference.).

As a result, as shown in FIG. 1, two NS-iPSC cell lines generated from the Noonan syndrome patient-derived fibroblasts were obtained (NS-iPSC #4 and NS-iPSC #5), from which the morphology of NS-iPSCs was normal (FIG. 1).

<2-2> Confirmation of Mutation of the Causing Gene of Noonan Syndrome in the Noonan Syndrome-Derived iPSCs

To investigate whether or not the Noonan syndrome-derived iPSCs could maintain the mutation of the causing gene of Noonan syndrome, the mutation of the causing gene of Noonan syndrome was investigated in NS-iPSCs.

Particularly, the NS-iPSCs induced by the same manner as described in Example <2-1> were obtained, from which genomic DNA (gDNA) was extracted. Mutated sequence of PTPN11 gene was investigated and compared with that of the Noonan syndrome-derived fibroblasts (NS-fibroblasts).

As a result, as shown in FIG. 2, the A922G mutation in PTPN11 gene, that is characteristically observed in the Noonan syndrome-derived fibroblasts, was observed in NS-iPSCs, suggesting that the mutation of the amino acid of N308D was confirmed in the protein originated therefrom (FIG. 2).

### Example 3

### Characteristics of the Noonan Syndrome-Derived iPSCs

### <3-1> Confirmation of Reprogramming Status in Promoter Regions of the Undifferentiated NS-iPSCs

To investigate whether or not the reprogramming progressed in the undifferentiated NS-iPSCs, bisulfite sequencing was performed and the demethylation of CpG in OCT4, REX1, and NANOG promoter regions of NS-iPSC was investigated by CT conversion (Park, S. W. et al. (2010) Blood 116, 5762-5772).

Particularly, genomic DNA was treated with sodium bisulfite by using EZ DNA methylation-Gold kit (Zymo Research, USA) according to the manufacturer's protocol. PCR amplification was performed by using 25˜50 ng of the bisulfite-treated DNA as a template. The amplified PCR product was purified by using AccuPrep plasmid Mini extraction Kit (Bioneer, Korea). It is followed by subcloning into pGEM-T EASY vector (Promega, USA). After transformation, the vectors were obtained. Sequencing was performed by using SP6, T7, and M13 primers, followed by analysis with Chromas231 program (Solgent, Korea).

As a result, as shown in FIG. 3, the demethylation was confirmed in the promoter region of each gene of the undifferentiated NS-iPSCs, compared with the phase of fibroblasts, suggesting that the reprogramming was completed in the NS-iPSCs (FIG. 3).

<3-2> Karyotyping of Noonan Syndrome-Derived iPSCs

To investigate whether or not there was any abnormality in the formation of chromosome in established NS-iPSCs, karyotype of the NS-iPSCs was analyzed to check insertion, transition, or deletion of chromosome.

Particularly, NS-iPSC #4 and NS-iPSC #5 induced by the same manner as described in Example <2-1> were obtained. The karyotypes of NS-iPSCs were analyzed by Gendix Co. (Korea).

As a result, as shown in FIG. 4, NS-iPSCs displayed normal karyotype (FIG. 4).

### <3-3> Confirmation of Undifferentiated Status in NS-iPSCs

To investigate whether the NS-iPSCs had undifferentiated status or not, the NS-iPSCs were stained with alkaline phosphatase, followed by observation of undifferentiation state.

Particularly, NS-iPSC #4 and NS-iPSC #5 induced by the same manner as described in Example <2-1> were prepared. 1 ml of citrate solution, 2.6 ml of acetone, and 320 μl of 37% formaldehyde (Sigma Aldrich, USA) provided in AP staining kit (alkaline phosphatase kit, Sigma Aldrich, USA) were mixed, leading to the preparation of fixative solution. Subsequently, 100 μl of sodium nitrate solution and 100 μl of FRV-alkaline solution were mixed together, which stood for 2 minutes. 4.5 ml of sterilized water and 100 μl of naphthol AS-BI alkali solution were added to the mixture above, which was rapped with foil to block the light. The prepared iPSCs were washed with PBS once and then added with the prepared fixative solution. After washing twice, AP staining solution was treated in fixed iPSCs. The mixture stood at room temperature for 15 minutes in the darkness. Thereafter, the mixture was washed with water or PBS twice, 2 minutes each time. The AP stained cells were observed under phase contrast microscope (Olympus, Japan).

As a result, as shown in FIG. 5, it was confirmed that the NS-iPSCs were maintained undifferentiated status (FIG. 5).

### Example 4

### Pluripotency of the Noonan Syndrome-Derived iPSCs

### <4-1> Pluripotent Gene Expression in NS-iPSCs

The expressions of pluripotent genes in NS-iPSCs were confirmed to investigate whether or not the undifferentiated NS-iPSCs had pluripotency.

Precisely, NS-iPSC #4 and NS-iPSC #5 induced by the same manner as described in Example <2-1> were suspended in Easy-blue (Intron, Korea). Total RNA was extracted from the NS-iPSCs according to the manufacturer′ protocol. Total cDNA was synthesized from 1 fig of the extracted RNA by using M-MLV reverse transcriptase (moloney-murine leukemia virus reverse transcriptase; Enzynomics, Korea). The synthesized total cDNA was amplified with the forward primer and the reverse primer listed in Table 4, followed by electrophoresis to confirm the gene expression at mRNA level. A human normal embryonic stem cell line (H9 hESC) was used as the normal control, and Noonan syndrome patient-derived fibroblasts were used as the negative control. The expressions of OCT4, SOX2, NANOG, c-MYC, KLF4, REX1, ECAT15, GDF3, and TERT were confirmed in the normal stem cell line by the same manner as described above. To correct the expression levels, the expression of GAPDH gene was measured by the same manner as the above, which was used as the control.

TABLE 4
Primer sequences to confirm expressions of pluripotent marker genes

| Gene | Primer name | Primer sequence | SEQ. ID. NO. |
|---|---|---|---|
| OCT4 | OCT4_F | TCGGGGTGGAGAGCAACT | SEQ. ID. NO: |
| | | | 1 |
| OCT4 | OCT4_R | GGGTGATCCTCTTCTGCTTC | SEQ. ID. NO: |
| | | | 2 |
| SOX2 | SOX2_F | ACTGGCGAACCATCTCTGTG | SEQ. ID. NO: |
| | | | 3 |
| SOX2 | SOX2_R | AATTACCAACGGTGTCAACCTG | SEQ. ID. NO: |
| | | | 4 |
| c-MYC | c-MYC_F | CCTACCCTCTCAACGACAGC | SEQ. ID. NO: |
| | | | 5 |
| c-MYC | c-MYC_R | CTCTGACCTTTTGCCAGGAG | SEQ. ID. NO: |
| | | | 6 |
| ECAT4- | ECAT4-macaca- | CAGCCCCGATTCTTCCACCAGTCC | SEQ. ID. NO: |
| macaca | 968S | C | 7 |
| ECAT4- | ECAT4-macaca- | CGGAAGATTCCCAGTCGGGTTCAC | SEQ. ID. NO: |
| macaca | 1334A5 | C | 8 |
| KLF4 | KLF4_F | GAACTGACCAGGCACTACCG | SEQ. ID. NO: |
| | | | 9 |
| KLF4 | KLF4_R | TTCTGGCAGTGTGGGTCATA | SEQ. ID. NO: |
| | | | 10 |
| hREX1 | hREX1-RT-U | CAGATCCTAAACAGCTCGCAGAAT | SEQ. ID. NO: |
| | | | 11 |
| hREX1 | hREX1-RT-L | GCGTACGCAAATTAAAGTCCAGA | SEQ. ID. NO: |
| | | | 12 |
| hECAT15-1 | hECAT15-1- | GGAGCCGCCTGCCCTGGAAAATTC | SEQ. ID. NO: |
| | S532 | | 13 |
| hECAT15-1 | hECAT15-1- | TTTTTCCTGATATTCTATTCCCAT | SEQ. ID. NO: |
| | AS916 | | 14 |
| hTERT | hTERT-F-3012 | TGTGCACCAACATCTACAAG | SEQ. ID. NO: |
| | | | 15 |
| hTERT | hTERT-R-3177 | GCGTTCTTGGCTTTCAGGAT | SEQ. ID. NO: |
| | | | 16 |
| GAPDH | GAPDH_F | CTTCGCTCTCTGCTCCTCCT | SEQ. ID. NO: |
| | | | 17 |
| GAPDH | GAPDH_R | GTTAAAAGCAGCCCTGGTGA | SEQ. ID. NO: |
| | | | 18 |

As a result, as shown in FIG. 6, the expressions of the pluripotent marker genes such as OCT4, SOX2, NANOG, c-MYC, REX1, ECAT15, GDF3, and TERT were not observed in the Noonan syndrome-derived fibroblasts, while the expressions of the pluripotent marker genes in NS-iPSCs were observed similar to H9 cell line (FIG. 6).

### <4-2> Confirmation of the Pluripotent Marker Protein Expression in NS-iPSCs

The expression of the stemness marker protein in NS-iPSCs was investigated in order to confirm whether the undifferentiated NS-iPSCs had pluripotency or not.

Particularly, NS-iPSC #4 and NS-iPSC #5 induced by the same manner as described in Example <2-1> were fixed in 4% formalin (formalin solution 10% neutral buffered, Sigma Aldrich, USA) at room temperature for 30 minutes. The cells were washed in PBS-T (PBS (phosphate buffered saline, GIBCO, USA) containing 0.1% Tween-20 (Sigma Aldrich, USA) three times, 10 minutes for each time. After washing, the cells were treated with PBS containing 0.1% triton X-100 at room temperature for 30 minutes to give permeability to the cell membrane. These iPSCs were treated with 3% BSA (bovine serum albumin, Sigma Aldrich, USA) at room temperature for 1 hour for blocking, followed by treatment with the primary antibody, anti-OCT4 antibody (1:200, Santa Cruz, USA), anti-SOX2 antibody (1:200, Cell Signaling Technology, USA), anti-NANOG antibody (1:200, Cell Signaling Technology, USA), anti-SSEA-4 antibody (1:200, R&D Systems, USA), anti-Tra-1-60 antibody (1:200, Millipore, USA), or anti-Tra-1-81 antibody (1:200, Millipore, USA). Then, the cells were incubated at 4° C. for overnight, followed by washing a few times with PBS-T. After washing, the cells were treated with the secondary antibody conjugated with Alexa Fluor 488 (Invitrogen, USA) or Alexa Fluor 594 (Invitrogen, USA), followed by incubation for 1 hour at room temperature. The cells were observed under fluorescence microscope (Olympus, Japan) to investigate the expressions of OCT4, SOX2, NANOG, SSEA-4, Tra-1-60, and Tra-1-81. To investigate whether or not expression of the protein was specific in the inside of the cell and in the outside of the cell, DAPI (4′6-diamidino-2-phenylindole, Sigma Aldrich, USA) was used to stain the nucleus of the cells.

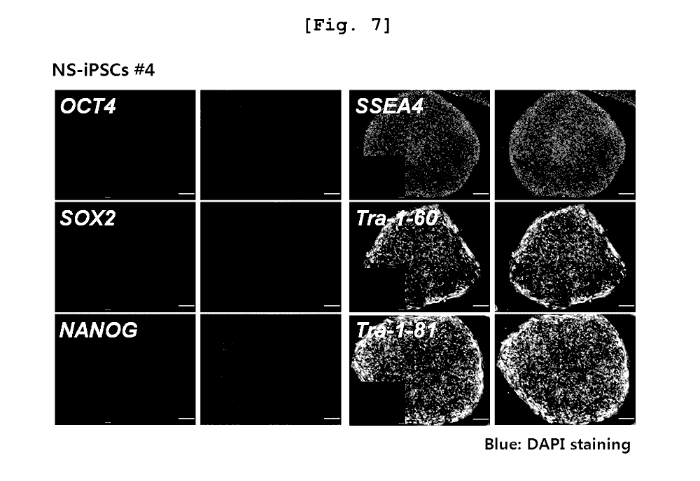

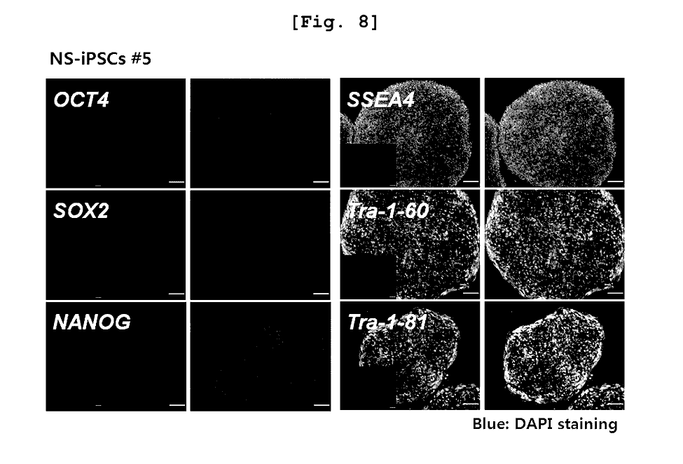

As a result, as shown in FIGS. 7 and 8, it was confirmed that the expressions of the stemness marker proteins such as OCT4, SOX2, NANOG, SSEA-4, Tra-1-60, and Tra-1-81 were significantly observed in NS-iPSCs (FIGS. 7 and 8).

<4-3> Differentiation Potency of the Noonan Syndrome-Derived iPSCs

To investigate if the Noonan syndrome-derived iPSCs had pluripotency in vivo, the teratoma formation of the NS-iPSCs was observed in the immunocompromised nude mouse.

Particularly, NS-iPSC #4 and NS-iPSC #5 induced by the same manner as described in Example <2-1> were prepared. These iPSCs placed on a 60 mm cell culture dish were mixed with matrigel (BD biosciences, USA). The prepared samples were injected in each 4 week old nude mouse (CAnN.Cg-Foxn1 nu/crlj0ri, female, OrientBio, Korea) by using 18 G syringe between the chest and the side, and then the animals were raised in the germ-free SPF animal lab. After 40 days of growing, the teratoma formed in the mouse was separated and washed with PBS twice or three times. The washed teratoma was added with 4% formaldehyde, followed by fixing at 4° C. for overnight. After fixing, the teratoma was loaded in a plastic cassette, and washed with running water for 6 hours to remove the fixative solution. Moisture in the tissue was eliminated by alcohol with increasing the concentration of alcohol from the low concentration to the high concentration. The tissue was fixed and embedded by paraffin infiltration. The fixed tissue was sectioned and the section was placed on the slide glass. Paraffin was eliminated, followed by hydration using alcohol with reducing the concentration from the high concentration to the low concentration. Harris hematoxylin and Eosin were treated on sectioned tissues to perform Hematoxylin & Eosin staining. The stained tissue was observed under phase contrast microscope (Olympus, Japan). As a result, the formation of neural tissue (ectoderm), secretory gland (endoderm), and smooth muscle and adipose tissue (mesoderm) were confirmed.

As a result, as shown in FIGS. 9 and 10, it was confirmed that neural tissue (ectoderm), secretory gland (endoderm), and smooth muscle and adipose tissue (mesoderm) were formed by the pluripotency of NS-iPSCs, suggesting that the NS-iPSCs had effective pluripotency (FIGS. 9 and 10).

### Example 5

### Natural Differentiation of Embryoid Bodies (EBs) and Neural Rosettes Originated from Noonan Syndrome Patient

<5-1> Natural Differentiation of Embryoid Bodies (EBs) and Neural Rosettes from NS-iPSCs

To confirm the differentiation potency of NS-iPSCs into neural cells in vitro, the natural differentiation of embryoid bodies (EBs) and neural rosettes from NS-iPSCs was induced according to the procedure described in the schematic diagram of FIG. 11 (FIG. 11).

Particularly, the colonies of NS-iPSC #4 and NS-iPSC #5 induced by the same manner as described in Example <2-1> were quartered by using 1 ml insulin syringe. The quartered NS-iPSCs were loaded on ultra-low attachment dish, which were resuspended in 4 ml of embryoid body differentiation media which was the DMED/F12 (Invitrogen, USA) containing 10% serum replacement (SR; SPL life sciences Co., Ltd., Korea). The resuspended NS-iPSCs were cultured for 4 days to induce the differentiation into NS-EBs. Then, the differentiated NS-EBs were obtained and cultured for 4˜5 days on the matrigel (1:50, BD biosciences, USA) coated dish in the neural rosette media which consists of DMEM/F12 supplemented with N2, B27 and 20 ng/ml of bFGF (FGF2, R&D systems, USA). The generated Noonan syndrome-derived neural rosettes were mechanically selected and placed on the fibronectin (BD biosciences, USA) coated dish, followed by culture in the neural rosette media for 4˜5 days. As for the normal control, the normal human embryonic stem cell line H9 (H9 hESC) and human skin fibroblast-derived iPSC (CRL12) were induced into EBs and neural rosettes by the same manner as described above.

As a result, as shown in FIG. 12, shape of NS-EBs at 2 day of differentiation in NS-iPSCs was similar to H9 cell line. However, the shape of NS-EBs became broken at 4 day of differentiation. The NS-EBs obtained at this time was induced to neural rosettes on the matrigel-coated dish. As a result, it was observed that distintive structures of NS-neural rosettes were not observed. Therefore, the normal differentiation of embryoid bodies (EBs) and neural rosettes from NS-iPSCs was failed (FIG. 12).

### <5-2> Expressions of the Pluripotent Marker Genes in the Noonan Syndrome-Derived Embryoid Body

To investigate whether or not the pluripotency was reduced in the NS-EBs that did not have a normal EBs morphology, the expressions of pluripotent marker genes was investigated in the NS-EBs.

Particularly, natural differentiation of NS-EBs was induced by the same manner as described in Example <5-1>, from which NS-EBs were obtained respectively on day 2, day 3, and day 4 after differentiation. Then, the obtained NS-EBs were suspended in Easy blue, followed by the extraction of total RNA according to the manufacturer's protocol. Total cDNA was synthesized from 1 μg of the extracted RNA by using M-MLV reverse transcriptase. Quantitative real-time PCR (q-PCR) was performed by using the synthesized cDNA as a template with 10 pmole of each forward primer and reverse primer as listed in Table 4 by using Bio-Rad CFX manager (Bio-Rad Laboratories, USA) using SyberGreen (Invitrogen, USA). The expressions of OCT4, SOX2, NANOG, and c-MYC genes were measured at mRNA level. GAPDH gene was used to normalize the expression level. Relative expression was calculated for comparison. ΔCt value of each gene was calculated by the difference between Ct value of GAPDH and Ct value of each gene.

NS-iPSCs were used as the experimental group. The human normal embryonic stem cell line (H9)-derived EBs (H9-EBs) was used as the normal control. The expressions of OCT4, SOX2, NANOG, and c-MYC genes were investigated. The expression levels were compared with those in NS-EBs and the results are presented by the fold change calculated by the mathematical formula 1 below.

Expression Fold=2^{-(s}^{^{Δ}}^{ct-c}^{^{Δ}}^{ct)} [Mathematical Formula 1]

SΔCt: ΔCt of each gene in NS-EBs; and

CΔCt: ΔCt of each gene in H9-ES

As a result, as shown in FIG. 13, the expression of the stem cell-related genes were reduced in NS-EBs during the natural differentiation, compared with the level of the normal control, suggesting that the differentiation potency was reduced (FIG. 13).

### <5-3> the Expression Levels of BMP and TGF-β Signal Transduction System Genes in the Noonan Syndrome Patient-Derived EBs

To investigate the expression levels of the signal transduction factors in the NS-EBs naturally differentiated from a Noonan syndrome patient, the expressions of the downstream genes (Id1, Id2, SMAD2 and SMAD3), which are related with BMP and TGF-β pathways were measured in the naturally differentiated NS-EBs.

Particularly, NS-EBs were naturally differentiated by the same manner as described in Example <5-1>, during which NS-EBs were respectively collected on day 2, day 3, and day 4 after the differentiation started. The obtained NS-EBs were treated by the same manner as described in Example <5-2>. The expression levels of the downstream genes (Id1 and Id2) of BMP signal transduction system, and the downstream genes (SMAD2 and SMAD3) of TGF-β signal transduction system were measured at mRNA level. For the confirmation, the forward primer and the reverse primer listed in Table 5 were used. NS-iPSCs were used as the experimental group, while the normal human embryonic stem cell line (H9-ES) was used as the normal control.

TABLE 5
Primer sequences for the confirmation of expressions
of singaling-related genes in NS-EBs

| | Primer | | |
|---|---|---|---|
| Gene | name | Primer sequence | SEQ. ID. NO. |
| Id1 | Id1-F | GTGCGCTGTCTGTCTGAG | SEQ. ID. NO: |
| | | | 19 |
| | Id1-R | CTGATCTCGCCGTTGAGG | SEQ. ID. NO: |
| | | | 20 |
| Id2 | Id2-F | CGTGAGGTCCGTTAGGAAAA | SEQ. ID. NO: |
| | | | 21 |
| | Id2-R | AATTCAGAAGCCTGCAAGGA | SEQ. ID. NO: |
| | | | 22 |
| SMAD2 | SMAD2-F | CTGGCGTCTACTGCATTTCC | SEQ. ID. NO: |
| | | | 23 |
| | SMAD2-R | CAGGACACCCAATTCCTTCA | SEQ. ID. NO: |
| | | | 24 |
| SMAD3 | SMAD3-F | CGATGTCCCCAGCACATAA | SEQ. ID. NO: |
| | | | 25 |
| | SMAD3-R | ATTGGAGGGGTCGGTGAA | SEQ. ID. NO: |
| | | | 26 |

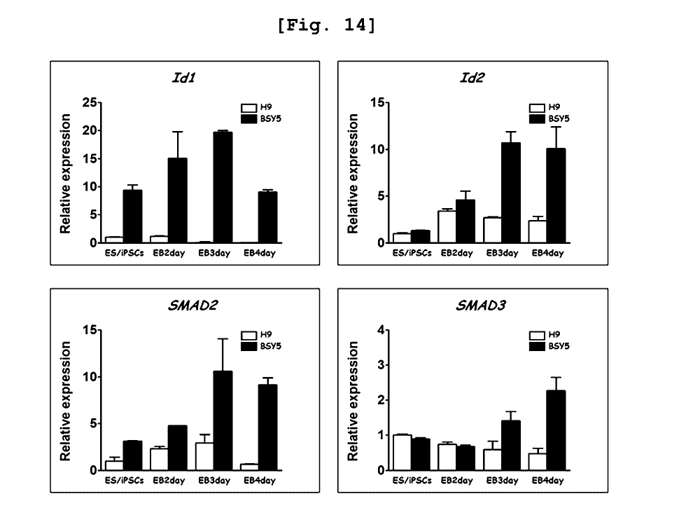

As a result, as shown in FIG. 14, the expressions of signaling-related genes in NS-EBs were significantly increased in the course of the natural differentiation, compared with those of the normal control (FIG. 14).

### <5-4> Phosphorylation Level of BMP Signal Transduction System Protein in the Noonan Syndrome Patient-Derived EBs

To investigate the expressions of signal transduction system factors in the NS-EBs naturally differentiated from a Noonan syndrome patient, phosphorylation levels of SMAD1/5/8 related with BMP pathway and SMAD2/3 mediating TGF-β pathway were measured in the naturally differentiated NS-EBs.

Particularly, the normal control H9 hESCs, CRL12 iPSCs (control) and NS-EBs #4 and NS-EBs #5 were naturally differentiated by the same manner as described in Example <5-1>, during which each NS-EBs was collected on day 2 and day 4 after the differentiation started. The NS-EBs were resuspended in RIPA cell lysis buffer 1× (R4100-100, GenDEPOT, USA) containing EDTA. The resuspended cells were lysed by vortexing on ice 3˜4 times at 10 minutes intervals, followed by centrifugation at 4° C., 13,000 rpm, for 30 minutes. The supernatant containing total protein of the cells was obtained, and the concentration of each protein was analyzed by using BCA protein assay kit (Thermo scientific, USA) with the standard curve. Each supernatant obtained from the cells was diluted in 60 mM Tris-HCl buffer (pH 6.8) containing 25% glycerol, 2% sodium dodecyl sulfate (SDS), 14.4 mM β-mercaptoethanol, and 0.1% bromophenol blue, followed by heating at 100° C. for 3 minutes. Heated proteins having the molecular weight of 30˜100 kDa were separated on 10% SDS-PAGE gel, which were transferred onto a nitrocellulose membrane, followed by blocking with 4% skim milk or 5% BSA (bovine serum albumin). After the blocking, the membrane was treated with the primary antibodies such as p-ERK1/2 (1:1000, #4370S, Cell signaling, USA), ERK1/2 (1:1000, #9102, Cell signaling, USA), SMAD2[5465/467] (1:500, #3108, Cell signaling, USA), p-SDMA2/3 (1:500, #3102, Cell signaling, USA), p-SMAD1/5/8 (1:500, #9511S, Cell signaling, USA), p-AKT[S473] (1:1000, #4051S, Cell signaling, USA), AKT (1:1000, #9272, Cell signaling, USA), and OCT4 (N-19, 1:1000, Santacruz, USA), followed by incubation at 4° C. for overnight. The membrane was then washed with TBS-T three times. The membrane was treated with the secondary antibodies such as goat-derived HRP (horseradish peroxidase) conjugated anti-rabbit IgG (Goat anti-Rabbit IgG (H+L) Secondary Antibody, HRP conjugate; Santacruz, USA), anti-mouse IgG (Goat anti-Rabbit IgG (H+L) Secondary Antibody, HRP conjugate; Santacruz, USA), and donkey-derived HRP (horseradish peroxidase) conjugated anti-goat IgG (Goat anti-Rabbit IgG (H+L) Secondary Antibody, HRP conjugate; Santacruz, USA) along with TBS-T containing 4% skim milk (BD sciences, USA) for one hour. After washing the membrane with TBS-T three times, bands were observed by using LAS-4000 (Fujifilm, Japan). As for the control, the human normal embryonic stem cell line H9 (H9 hESC) was used. As for the normal control, the human skin fibroblast-derived iPSCs (CRL12) were used. The differentiation of them was induced into EBs by the same manner as described above. To normalize the expressions, actin (actin-HRP conjugate, Santacruz, USA) was used as the housekeeping protein.

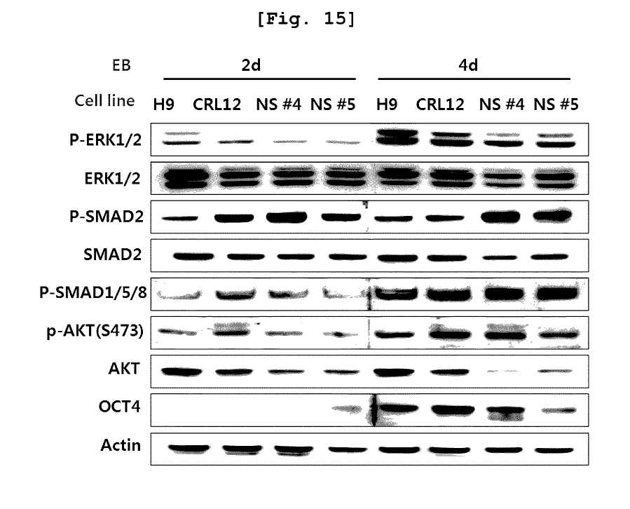

As a result, as shown in FIG. 15, the expressions of p-SMAD1/5/8 related with BMP signaling and p-SMAD2/3 mediating TGF-β signaling were significantly increased on day 2 and day 4, compared with the normal control (FIG. 15).

### <5-5> Expression of Neural Marker Genes in the Noonan Syndrome-Derived Neural Rosettes

To investigate whether or not the neural rosettes naturally differentiated from NS-EBs displayed the characteristics of neural cells, the mRNA expression levels of PAX6, SOX2, ZIC1, OTX2, and NESTIN genes in the Noonan syndrome-derived neural rosettes were measured.

Particularly, the Noonan syndrome-derived neural rosettes (NS-NR) differentiated by the same manner as described in Example <5-1> were obtained. cDNAs including PAX6, SOX2, ZIC1, OTX2, and NESTIN genes were synthesized by the same manner as described in Example <5-2>. Each of the synthesized cDNA was amplified, followed by electrophoresis to investigate the expression of each gene above at mRNA level. As the control group for the normalization of the expression level of each gene above in RT-PCR and qPCR, GAPDH gene was used so that the expression was also measured. ΔCt value of each gene was calculated by the difference between Ct value of GAPDH and Ct value of each gene. As for the control, the human normal embryonic stem cell line H9 and the human normal fibroblast-derived iPSCs (CRL12) were induced into neural rosettes (H9-NR and CRL12-NR) via natural differentiation as described in Example <5-1>. The expressions of those NRs were compared with the gene expression of NS-neural rosettes. The results are presented by the fold change calculated by the mathematical formula 1 above.

As a result, as shown in FIGS. 16 and 17, it was confirmed that the neural rosettes and neural tube were not formed in NS-iPSCs. The expression of SOX2 associated with the above was significantly reduced and the expressions of PAX6, OTX2, and ZIC1 were hardly observed. In the meantime, the expression of NESTIN was not changed (FIGS. 16 and 17).

### Example 6

### Chemically Directed Differentiation of Embryoid Bodies (EBs) and Neural Rosettes Originated from Noonan Syndrome Patient

<6-1> Chemically Directed Differentiation of Embryoid Bodies (EBs) and Neural Rosettes from NS-iPSCs

Due to the abnormality in the signal transduction system in Noonan syndrome, EBs and neural rosettes naturally differentiated from NS-iPSCs were abnormally developed. To overcome this problem, the present inventors induced chemically directed differentiation of EBs and neural rosettes from NS-iPSCs by using dorsomorphin (DM) and SB431542 according to the schematic diagram of FIG. 18 (FIG. 18 and Table 6).

Particularly, the colonies of NS-iPSC #4 and NS-iPSC #5 induced by the same manner as described in Example <2-1> were quartered by using 1 ml insulin syringe. The quartered NS-iPSCs were loaded on ultra-low attachment dish (SPL life sciences Co., Ltd, Korea), which were resuspended in 4 ml of embryoid body differentiation media which was the DMEM/F12 (Invitrogen, USA) containing 10% serum replacement (SR; GIBCO, USA). The resuspended NS-iPSCs were cultured for 4 days to induce the differentiation into NS-EBs. Then, the differentiated NS-EBs were obtained and cultured for 4˜5 days on the matrigel (1:50, BD biosciences, USA) coated dish in the neural rosette media which consists of DMEM/F12 supplemented with N2, B27 and 20 ng/a of bFGF (FGF2, R&D systems, USA). The generated Noonan syndrome-derived neural rosettes were mechanically selected and placed on the fibronectin (BD biosciences, USA) coated dish, followed by culture in the neural rosette media for 4˜5 days. As for the normal control, EBs differentiation was induced from the normal human embryonic stem cell line H9 (H9 hESC) and human skin fibroblast-derived iPSC (CRL12) by the same manner as described above.

TABLE 6
Composition of EBs differentiation media for the
inducement of chemically directed differentiation

| Day | Composition |
|---|---|
| 1 | DMEM/F12 containing 10% SR* |
| 2 | DMEM/F12 containing 10% SR and 10 μM |
| | SB431542 |
| 3 | DMEM/F12 containing 10% SR, 10 μM |
| | SB431542, and 5 μM DM |
| 4 | N2** containing 20 ng/ml bFGF |

*SR: serum replacement
**N2 medium was added on the matrigel coated dish.

As a result, as shown in FIGS. 19 and 20, compared with the NS-EBs and NS-NR induced via natural differentiation, chemically differentiated NS-EBs and NS-NR displayed the recovery of cell morphology almost as similar as the normal shape observed in the normal control, suggesting that neural rosettes and neural tube were successfully formed (FIGS. 19 and 20).

<6-2> Gene Xpressions of Neural Markers in the Neural Rosettes Chemically Differentiated from NS-iPSC

To investigate whether or not the neural marker genes were normally expressed in the neural rosettes chemically differentiated from NS-iPSCs, the mRNA expression levels of the ectoderm genes PAX6, ZIC1, NESTIN, and VIMENTIN, and the NR genes PLZF, NR2F1, HES5, and DACH1, and the neural genes TUJ1, ASCL1, and NF1 were measured in the chemically differentiated NS-EBs.

Particularly, the neural rosettes chemically differentiated from NS-iPSCs by the same manner as described in Example <6-1> were prepared. Total RNA was extracted from NS-NR by the same manner as described in Example <5-2>. cDNA was synthesized from 1 fig of the extracted RNA using M-MLV reverse transcriptase (Enzynomics, Korea). cDNAs were amplified with the primers listed in Table 7. The expressions of those genes were observed by RT-PCR at mRNA level. As for the normal control, the neural rosettes chemically differentiated from H9 cell line and CRL12 in Example <6-1> were used.

TABLE 7
Primer sequences for the confirmation of the neural marker
gene expression in neural rosette

| | Primer | | |
|---|---|---|---|
| Gene | name | Primer sequence | SEQ. ID. NO. |
| PTPN11 | PTPN11-F | CTGTGCAGATCCTACCTCTGA | SEQ. ID. NO: |
| | | | 27 |
| | PTPN11-R | TCCCCTTTGTCATCACCAGT | SEQ. ID. NO: |
| | | | 28 |
| PAX6 | PAX6_F | GTGTCCAACGGATGTGTGAG | SEQ. ID. NO: |
| | | | 29 |
| | PAX6_R | CTAGCCAGGTTGCGAAGAAC | SEQ. ID. NO: |
| | | | 30 |
| ZIC1 | ZIC1-F | GCGCTCCGAGAATTTAAAGA | SEQ. ID. NO: |
| | | | 31 |
| | ZIC1-R | CGTGGACCTTCATGTGTTTG | SEQ. ID. NO: |
| | | | 32 |
| NESTIN | NESTIN-F | GCAGGAGAAACAGGGCCTAC | SEQ. ID. NO: |
| | | | 33 |
| | NESTIN-R | AAAGCTGAGGGAAGTCTTGGA | SEQ. ID. NO: |
| | | | 34 |
| VIMENTIN | VIMENTIN- | TGCAGGACTCGGTGGACTT | SEQ. ID. NO: |
| | F | | 35 |
| | VIMENTIN- | TGGACTCCTGCTTTGCCTG | SEQ. ID. NO: |
| | R | | 36 |
| PLZF | PLZF-F | CTATGGGCGAGAGGAGAGTG | SEQ. ID. NO: |
| | | | 37 |
| | PLZF-R | TCAATACAGCGTCAGCCTTG | SEQ. ID. NO: |
| | | | 38 |
| NR2F1 | NR2F1-F | ACAGGAACTGTCCCATCGAC | SEQ. ID. NO: |
| | | | 39 |
| | NR2F1-R | GATGTAGCCGGACAGGTAGC | SEQ. ID. NO: |
| | | | 40 |
| HES5 | HESS-F | CATCCTGGAGATGGCTGTCA | SEQ. ID. NO: |
| | | | 41 |
| | HESS-R | AGCAGCTTCATCTGCGTGT | SEQ. ID. NO: |
| | | | 42 |
| DACH1 | DACH1-F | GTGGAAAACACCCCTCAGAA | SEQ. ID. NO: |
| | | | 43 |
| | DACH1-R | CTTGTTCCACATTGCACACC | SEQ. ID. NO: |
| | | | 44 |
| TUJ1 | TUJ1-F | GAACAGCACGGCCATCCAGG | SEQ. ID. NO: |
| | | | 45 |
| | TUJ1-R | CTTGGGGCCCTGGGCCTCCGA | SEQ. ID. NO: |
| | | | 46 |
| ASCL1 | ASCL1-F | CATCTCCCCCAACTACTCCA | SEQ. ID. NO: |
| | | | 47 |
| | ASCL1-R | TCGGGGCTGAGCGGGTCGTA | SEQ. ID. NO: |
| | | | 48 |
| NF1 | NF1-F | AGCAGCAGTTTGGCCACTACA | SEQ. ID. NO: |
| | | A | 49 |
| | NF1-R | TTGCAGCACTTTCTGTCAGCT | SEQ. ID. NO: |
| | | G | 50 |

As a result, as shown in FIG. 21, most of the genes such as PAX6, ZIC1, NESTIN, VIMENTIN, PLZF, HES5, DACH1, TUJ1, ASCL1, and NF1 were expressed similarly to those shown in the normal control, but the expression of NR2F1 was decreased (FIG. 21).

<6-3> Decreased Expression of the NR2F1 Gene in the Neural Rosette Chemically Differentiated from NS-iPSC

To investigate whether or not NR2F1 gene was down-regulated in the neural rosettes chemically differentiated from NS-iPSC, the expression of NR2F1 gene was re-confirmed quantitatively.

Particularly, the neural rosette chemically differentiated from NS-iPSC by the same manner as described in Example <6-1> was obtained. cDNA containing NR2F1 gene was synthesized by the same manner as described in Example <5-2>. The expression of NR2F1 gene was measured by q-PCR at mRNA level. As the control group for the normalization of the expression level, GAPDH gene was used so that the expression was also measured. ΔCt value of each gene was calculated by the difference between Ct value of GAPDH and Ct value of NR2F1 gene. As for the control, the human normal embryonic stem cell line H9 and the human normal fibroblast-derived iPSCs (CRL12) were induced into neural rosettes via chemical treatments as described in Example <6-1>. The expressions of those neural rosettes were compared with the gene expression of NS-NR. The results are presented by the fold change calculated by the mathematical formula 1 above.

As a result, as shown in FIG. 22, it was confirmed that the expression level of NR2F1 gene in NS-NR was significantly reduced, compared with the level of the normal control (FIG. 22).

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended Claims.

1-12. (canceled)

13. A method for using the iPSC as a Noonan syndrome model, which comprises the following steps:
i) inducing the differentiation of the induced pluripotent stem cells (iPSCs) from the fibroblasts separated from Noonan syndrome patients into embryoid bodies (EBs) in vitro; and
ii) analyzing the characteristics of the embryoid bodies differentiated in step i), wherein the characteristics are the following a)˜f):
a) normal morphology of EBs;
b) the expression of one or more pluripotent marker genes selected from the group consisting of OCT4, SOX2, NANOG, c-MYC and KLF4;
c) the increased expression of BMP signaling genes including one of or both of Id1 and Id2 when compared with the embryoid bodies from the normal cell-derived iPSCs;
d) the increased phosphorylation level of one or more BMP signaling proteins selected from the group consisting of p-SMAD1, p-SMAD5, and p-SMAD8 when compared with the embryoid bodies from the normal cell-derived iPSCs;
e) the increased expression of TGF-β signaling genes including one of or both of SMAD2 and SMAD3 when compared with the embryoid bodies from the normal cell-derived iPSCs; and
f) the increased phosphorylation level of TGF-β signaling proteins including one of or both of p-SMAD2 and p-SMAD3 when compared with the embryoid bodies from the normal cell-derived iPSCs.

14. The method for using the iPSC as a Noonan syndrome model according to claim 13, wherein the differentiation is induced either naturally or chemically directed.

15. The method for using the iPSC as a Noonan syndrome model according to claim 13, wherein the characteristics of the iPSC model of step ii) are analyzed by investigating the differentiation potency of the iPSC model into embryoid bodies or neural cells.

16. A method for using the iPSC as a Noonan syndrome model, which comprises the following steps:
i) inducing the differentiation of the induced pluripotent stem cells (iPSCs) from the fibroblasts separated from a Noonan syndrome patient into neural cells in vitro; and
ii) analyzing the characteristics of the neural cells differentiated in step i), wherein the characteristics are following a)˜c):
a) normal morphology of neural cells;
b) the expression of one or more neural genes selected from the group consisting of PAX6, ZIC1, NESTIN, VIMENTIN, PLZF, HES5, DACH1, TUJ1, ASCL1, and NF1; and
c) the decreased expression of the NR2F1 gene, compared with the normal cell.

17. The method for using the iPSC as a Noonan syndrome model according to claim 16, wherein the differentiation is induced either naturally or chemically directed.

18. The method for using the iPSC as a Noonan syndrome model according to claim 16, wherein the characteristics of the iPSC model of step ii) are analyzed by investigating the differentiation potency of the iPSC model into embryoid bodies or neural cells.

19. A method for screening a therapeutic agent candidate for Noonan syndrome comprising the following steps:
i) obtaining embryoid bodies or neural cells differentiated from the prepared iPSC model;
ii) treating the test compound or the test composition to the embryoid bodies or neural cells of step i);
iii) analyzing a characteristics of the embryoid bodies or a level of NF2R1 gene expression of the neural cells treated in step ii); and
iv) comparing the results of the analysis of step iii) with the non-treated control, and selecting the test compound or the test composition which induce characteristics of embryoid body of the following a)˜d):
a) the increased expression of BMP signaling genes including one of or both of Id1 and Id2 when compared with the non-treated control;
b) the increased phosphorylation level of one or more BMP signaling proteins selected from the group consisting of p-SMAD1, p-SMAD5, and p-SMAD8 when compared with the non-treated control;
c) the increased expression of TGF-β signaling genes including one of or both of SMAD2 and SMAD3 when compared with the non-treated control; and
d) the increased phosphorylation level of TGF-β signaling proteins including one of or both of p-SMAD2 and p-SMAD3 when compared with the non-treated control, or which induce more higher expression of the NR2F1 gene in the treated neural cells than the non-treated control.

20. The method according to claim 19 comprising:
i) obtaining embryoid bodies differentiated from the prepared iPSC model;
ii) treating the test compound or the test composition to the embryoid bodies of step i);
iii) analyzing the characteristics of the embryoid bodies treated in step ii); and
iv) comparing the results of the analysis of step iii) with the non-treated control, and selecting the test compound or the test composition which induce characteristics of embryoid body of the following a)˜d):
a) the increased expression of BMP signaling genes including one of or both of Id1 and Id2 when compared with the non-treated control;
b) the increased phosphorylation level of one or more BMP signaling proteins selected from the group consisting of p-SMAD1, p-SMAD5, and p-SMAD8 when compared with the non-treated control;
c) the increased expression of TGF-β signaling genes including one of or both of SMAD2 and SMAD3 when compared with the non-treated control; and
d) the increased phosphorylation level of TGF-β signaling proteins including one of or both of p-SMAD2 and p-SMAD3 when compared with the non-treated control.

21. The method according to claim 19 comprising:
i) obtaining neural cells differentiated from the prepared iPSC model;
ii) treating the test compound or the test composition to the neural cells of step i);
iii) analyzing the level of the NR2F1 gene expression of the neural cells treated in step ii); and
iv) comparing the results of the analysis of step iii) with the non-treated control, and selecting the test compound or the test composition which induce more higher expression of the NR2F1 gene in the treated neural cells than the non-treated control.


SEQUENCE LISTING (50 sequences)

SEQ ID NO: 1 - DNA, 18 bp, Artificial
  primer
tcggggtggagagcaact

SEQ ID NO: 2 - DNA, 20 bp, Artificial
  primer
gggtgatcctcttctgcttc

SEQ ID NO: 3 - DNA, 20 bp, Artificial
  primer
actggcgaaccatctctgtg

SEQ ID NO: 4 - DNA, 22 bp, Artificial
  primer
aattaccaacggtgtcaacctg

SEQ ID NO: 5 - DNA, 20 bp, Artificial
  primer
cctaccctctcaacgacagc

SEQ ID NO: 6 - DNA, 20 bp, Artificial
  primer
ctctgaccttttgccaggag

SEQ ID NO: 7 - DNA, 25 bp, Artificial
  primer
cagccccgattcttccaccagtccc

SEQ ID NO: 8 - DNA, 25 bp, Artificial
  primer
cggaagattcccagtcgggttcacc

SEQ ID NO: 9 - DNA, 20 bp, Artificial
  primer
gaactgaccaggcactaccg

SEQ ID NO: 10 - DNA, 20 bp, Artificial
  primer
ttctggcagtgtgggtcata

SEQ ID NO: 11 - DNA, 24 bp, Artificial
  primer
cagatcctaaacagctcgcagaat

SEQ ID NO: 12 - DNA, 23 bp, Artificial
  primer
gcgtacgcaaattaaagtccaga

SEQ ID NO: 13 - DNA, 24 bp, Artificial
  primer
ggagccgcctgccctggaaaattc

SEQ ID NO: 14 - DNA, 24 bp, Artificial
  primer
tttttcctgatattctattcccat

SEQ ID NO: 15 - DNA, 20 bp, Artificial
  primer
tgtgcaccaacatctacaag

SEQ ID NO: 16 - DNA, 20 bp, Artificial
  primer
gcgttcttggctttcaggat

SEQ ID NO: 17 - DNA, 20 bp, Artificial
  primer
cttcgctctctgctcctcct

SEQ ID NO: 18 - DNA, 20 bp, Artificial
  primer
gttaaaagcagccctggtga

SEQ ID NO: 19 - DNA, 18 bp, Artificial
  primer
gtgcgctgtctgtctgag

SEQ ID NO: 20 - DNA, 18 bp, Artificial
  primer
ctgatctcgccgttgagg

SEQ ID NO: 21 - DNA, 20 bp, Artificial
  primer
cgtgaggtccgttaggaaaa

SEQ ID NO: 22 - DNA, 20 bp, Artificial
  primer
aattcagaagcctgcaagga

SEQ ID NO: 23 - DNA, 20 bp, Artificial
  primer
ctggcgtctactgcatttcc

SEQ ID NO: 24 - DNA, 20 bp, Artificial
  primer
caggacacccaattccttca

SEQ ID NO: 25 - DNA, 19 bp, Artificial
  primer
cgatgtccccagcacataa

SEQ ID NO: 26 - DNA, 18 bp, Artificial
  primer
attggaggggtcggtgaa

SEQ ID NO: 27 - DNA, 21 bp, Artificial
  primer
ctgtgcagatcctacctctga

SEQ ID NO: 28 - DNA, 20 bp, Artificial
  primer
tcccctttgtcatcaccagt

SEQ ID NO: 29 - DNA, 20 bp, Artificial
  primer
gtgtccaacggatgtgtgag

SEQ ID NO: 30 - DNA, 20 bp, Artificial
  primer
ctagccaggttgcgaagaac

SEQ ID NO: 31 - DNA, 20 bp, Artificial
  primer
gcgctccgagaatttaaaga

SEQ ID NO: 32 - DNA, 20 bp, Artificial
  primer
cgtggaccttcatgtgtttg

SEQ ID NO: 33 - DNA, 20 bp, Artificial
  primer
gcaggagaaacagggcctac

SEQ ID NO: 34 - DNA, 21 bp, Artificial
  primer
aaagctgagggaagtcttgga

SEQ ID NO: 35 - DNA, 19 bp, Artificial
  primer
tgcaggactcggtggactt

SEQ ID NO: 36 - DNA, 19 bp, Artificial
  primer
tggactcctgctttgcctg

SEQ ID NO: 37 - DNA, 20 bp, Artificial
  primer
ctatgggcgagaggagagtg

SEQ ID NO: 38 - DNA, 20 bp, Artificial
  primer
tcaatacagcgtcagccttg

SEQ ID NO: 39 - DNA, 20 bp, Artificial
  primer
acaggaactgtcccatcgac

SEQ ID NO: 40 - DNA, 20 bp, Artificial
  primer
gatgtagccggacaggtagc

SEQ ID NO: 41 - DNA, 20 bp, Artificial
  primer
catcctggagatggctgtca

SEQ ID NO: 42 - DNA, 19 bp, Artificial
  primer
agcagcttcatctgcgtgt

SEQ ID NO: 43 - DNA, 20 bp, Artificial
  primer
gtggaaaacacccctcagaa

SEQ ID NO: 44 - DNA, 20 bp, Artificial
  primer
cttgttccacattgcacacc

SEQ ID NO: 45 - DNA, 20 bp, Artificial
  primer
gaacagcacggccatccagg

SEQ ID NO: 46 - DNA, 21 bp, Artificial
  primer
cttggggccctgggcctccga

SEQ ID NO: 47 - DNA, 20 bp, Artificial
  primer
catctcccccaactactcca

SEQ ID NO: 48 - DNA, 20 bp, Artificial
  primer
tcggggctgagcgggtcgta

SEQ ID NO: 49 - DNA, 22 bp, Artificial
  primer
agcagcagtttggccactacaa

SEQ ID NO: 50 - DNA, 22 bp, Artificial
  primer
ttgcagcactttctgtcagctg